(19) United States
(12) Patent Application Publication
(10) Pub. No.: US 2009/0192227 A1
(43) Pub. Date: Jul. 30, 2009

(54) N-Acetylcysteine Compositions and Methods for Treating Acute Exacerbations of Inflammatory Lung Disease

(76) Inventors: Rabindra Tirouvanziam, Stanford, CA (US); Leonore A. Herzenberg, Stanford, CA (US)

Correspondence Address: GREENBERG TRAURIG, LLP, 200 PARK AVE., P.O. BOX 677, FLORHAM PARK, NJ, 07932, US

(21) Appl. No.: 12/420,577

(22) Filed: Apr. 8, 2009

Related U.S. Application Data
(63) Continuation-in-part of application No. 11/507,706, filed on Aug. 22, 2006.
(60) Provisional application No. 61/044,943, filed on Apr. 15, 2008.
(60) Provisional application No. 60/710,807, filed on Aug. 24, 2005.

(51) Int. Cl. A61K 31/195; A61P 11/12

(52) U.S. Cl. 514/562

(57) ABSTRACT

The present invention relates to N-acetylcysteine compositions and methods for treating inflammation and redox imbalance in acute exacerbations of inflammatory lung disease.

### CROSS-REFERENCE TO RELATED APPLICATIONS

This application claims the benefit of priority to U.S. Application No. 61/044,943 (filed Apr. 15, 2008) and is a continuation-in part of U.S. application Ser. No. 11/507,706 (filed Aug. 22, 2006), which claims the benefit of priority to U.S. Provisional Application No. 60/710,807 (filed Aug. 24, 2005) entitled “Methods For Treating And Monitoring Inflammation And Redox Imbalance In Cystic Fibrosis.” The entire contents of each of these applications are incorporated herein by reference.

### FIELD OF THE INVENTION

The present invention relates to N-acetylcysteine compositions and methods for treating inflammation and redox imbalance in acute exacerbations of inflammatory lung disease.

### BACKGROUND OF THE INVENTION

### Oxidative Stress Associated with GSH Depletion

A free radical is a highly reactive and usually short-lived molecular fragment with one or more unpaired electrons. Free radicals are highly chemically reactive molecules. Because a free radical needs to extract a second electron from a neighboring molecule to pair its single electron, it often reacts with other molecules, which initiates the formation of many more free radical species in a self-propagating chain reaction. This ability to be self-propagating makes free radicals highly toxic to living organisms.

Living systems under normal conditions produce the vast majority of free radicals and free radical intermediates. They handle free radicals formed by the breakdown of compounds through the process of metabolism. Most reactive oxygen species come from endogenous sources as by-products of normal and essential metabolic reactions, such as energy generation from mitochondria or detoxification reactions involving the cytochrome P-450 enzyme system. The major sources of free radicals, such as O₂⁻ and HNO₂⁻, are modest leakages from the electron transport chains of mitochondria, chloroplasts, and endoplasmic reticulum.

Reactive oxygen species (“ROS”), such as free radicals and peroxides, represent a class of molecules that are derived from the metabolism of oxygen and exist inherently in all aerobic organisms. The term “oxygen radicals” as used herein refers to any oxygen species that carries an unpaired electron (except free oxygen). The transfer of electrons to oxygen also may lead to the production of toxic free radical species. The best documented of these is the superoxide radical. Oxygen radicals, such as the hydroxyl radical (OH—) and the superoxide ion (O₂⁻) are very powerful oxidizing agents that cause structural damage to proteins, lipids and nucleic acids. The free radical superoxide anion, a product of normal cellular metabolism, is produced mainly in mitochondria because of incomplete reduction of oxygen. The superoxide radical, although unreactive compared with many other radicals, may be converted by biological systems into other more reactive species, such as peroxyl (ROO⁻), alkoxyl (RO⁻) and hydroxyl (OH⁻) radicals.

The major cellular sources of free radicals under normal physiological conditions are the mitochondria and inflammatory cells, such as granulocytes, macrophages, and some T-lymphocytes, which produce active species of oxygen via the nicotinamide adenine nucleotide oxidase (NADPH oxidase) system, as part of the body's defense against bacterial, fungal or viral infections.

Oxidative injury may lead to widespread biochemical damage within the cell. The molecular mechanisms responsible for this damage are complex. For example, free radicals may damage intracellular macromolecules, such as nucleic acids (e.g., DNA and RNA), proteins, and lipids. Free radical damage to cellular proteins may lead to loss of enzymatic function and cell death. Free radical damage to DNA may cause problems in replication or transcription, leading to cell death or uncontrolled cell growth. Free radical damage to cell membrane lipids may cause the damaged membranes to lose their ability to transport oxygen, nutrients or water to cells.

Biological systems protect themselves against the damaging effects of activated species by several means. These include free radical scavengers and chain reaction terminators; “solid-state” defenses, and enzymes, such as superoxide dismutase, catalase, and the glutathione peroxidase system.

Free radical scavengers/chemical antioxidants, such as vitamin C and vitamin E, counteract and minimize free radical damage by donating or providing unpaired electrons to a free radical and converting it to a nonradical form. Such reducing compounds may terminate radical chain reactions and reduce hydroperoxides and epoxides to less reactive derivatives.

The term “solid state defense” as used herein refers to the mechanism whereby a macromolecule binds a radical-generating compound, de-excites an excited state species, or quenches a free radical. The most important solid-state defense in the body is the black pigment melanin, which scavenges odd electrons to form stable radical species, thus terminating radical chain reactions.

Enzymatic defenses against active free radical species include superoxide dismutase, catalases, and the glutathione reductase/peroxidase system. Superoxide dismutase (SOD) is an enzyme that destroys superoxide radicals. Catalase, a heme-based enzyme that catalyses the breakdown of hydrogen peroxide into oxygen and water, is found in all living cells, especially in the peroxisomes, which, in animal cells, are involved in the oxidation of fatty acids and the synthesis of cholesterol and bile acids. Hydrogen peroxide is a byproduct of fatty acid oxidation and is produced by white blood cells to kill bacteria.

Glutathione, a tripeptide composed of glycine, glutamic acid, and cysteine that contains a nucleophilic thiol (SH) group, is widely distributed in animal and plant tissues. It exists in both the reduced thiol form (GSH) and the oxidized disulfide form (GSSG). In its reduced GSH form, glutathione acts as a substrate for the enzymes GSH-S-transferases and GSH peroxidases, both of which catalyze reactions for the detoxification of xenobiotic compounds, and for the reduction of reactive oxygen species and other free radicals. The term “xenobiotic” is used herein to refer to a chemical which is not a natural component of the organism exposed to it.

Examples of xenobiotics include, but are not limited to, carcinogens, toxins and drugs. The metabolism of xenobiotics usually involves two distinct stages. Phase I metabolism involves an initial oxidation, reduction or dealkylation of the xenobiotic by microsomal cytochrome P-450 monooxygenases (Guengerich, F. P. Chem. Res. Toxicol. 4: 391-407 (1991); this step is often needed to provide hydroxyl- or amino groups, which are essential for phase II reactions. Glutathione detoxifies many highly reactive intermediates produced by cytochrome P450 enzymes in phase I metabolism. Without adequate GSH, the reactive toxic metabolites produced by cytochrome P-450 enzymes may accumulate causing organ damage.

Phase II metabolism generally adds hydrophilic moieties, thereby making a toxin more water soluble and less biologically active. Frequently involved phase II conjugation reactions are catalyzed by glutathione S-transferases (Beckett, G. J. & Hayes, J. D., Adv. Clin. Chem. 30: 281-380 (1993), sulfotransferases (Falany, C N, Trends Pharmacol. Sci. 12: 255-59 (1991), and UDP-glucuronyl-transferases (Bock, K W, Crit. Rev. Biochem. Mol. Biol. 26: 129-50 (1991). Glutathione S-transferases catalyze the addition of aliphatic, aromatic, or heterocyclic radicals as well as epoxides and arene oxides to glutathione. These glutathione conjugates then are cleaved to cysteine derivatives primarily by renal enzymes and then acetylated, thus forming N-acetylcysteine derivatives. Examples of compounds transformed to reactive intermediates and then bound to GSH include, but are not limited to, bromobenzene, chloroform, and acetaminophen. Such toxicants may deplete GSH.

Depletion of GSH may diminish the body's ability to defend against lipid peroxidation. Glutathione is a cofactor for Glutathione peroxidase (GPx), an enzyme of the oxidoreductase class, which catalyzes the detoxifying reduction of hydrogen peroxide and organic peroxides via oxidation of glutathione. GSH is oxidized to the disulfide linked dimer (GSSG), which is actively pumped out of cells and becomes largely unavailable for reconversion to reduced glutathione. Loss of large amounts of GSH results in cell death, while loss of smaller amounts can change cell function.

The generation of cytokine-induced neutrophil chemoattractants that affect neutrophil migration is induced in part by the nuclear factor KB (NF-κB) family of proteins, a set of transcription factors that lie at the heart of most inflammatory responses. Two vertebrate cytokines are especially important in inducing inflammatory responses—tumor necrosis factor α (TNF-α) and interleukin-1 (IL-1). Both of these proinflammatory cytokines, which are made by cells of the innate immune system, bind to cell surface receptors and activate NF-κB, which normally is sequestered in an inactive form in the cytoplasm of almost all cells. Once activated, NF-κB turns on the transcription of more than 60 known genes that participate in inflammatory responses, including the canonical neutrophil chemoattractant interleukin-8 (IL-8). NF-κB is responsive to the oxidative stress associated with GSH depletion.

Thus, unless glutathione is resynthesized through other pathways, utilization of oxidized glutathione is associated with a decrease in the amount of glutathione available.

Glutathione reductase, a flavoprotein enzyme of the oxidoreductase class, is essential for the maintenance of cellular glutathione in its reduced form (Carlberg & Mannervick, J. Biol. Chem. 250: 5475-80 (1975)). It catalyzes the reduction of oxidized glutathione (GSSG) to reduced glutathione (GSH) in the presence of NADPH and maintains a high intracellular GSH/GSSG ratio of about 500 in red blood cells.

Synthesis of GSH requires cysteine, a conditionally essential amino acid that must be obtained from dietary sources or by conversion of dietary methionine via the cystathionase pathway. If the supply of cysteine is adequate, normal GSH levels are maintained. But GSH depletion occurs if supplies of cysteine are inadequate to maintain GSH homeostasis in the face of increased GSH consumption. Acute GSH depletion causes severe—sometimes fatal—oxidative and/or alkylation injury, and chronic or slow arising GSH deficiency due to administration of GSH-depleting drugs, such as acetaminophen, or to diseases and conditions that deplete GSH, may be similarly debilitating.

Cysteine is necessary to replenish GSH. Although various forms of cysteine and its precursors have been used as nutritional and therapeutic sources of cysteine, N-acetylcysteine (NAC) is the most widely used and extensively studied. NAC is about 10 times more stable than cysteine and much more soluble than the stable cysteine disulfide, cystine. Glutathione, glutathione monoethyl ester, and L-2-oxothiazolidine-4-carboxylate (procysteine/OTC) also have been used effectively in some studies. In addition, dietary methionine and S-adenosylmethionine are an effective source of cysteine.

It is well known that NAC, as a cysteine prodrug, promotes cellular glutathione production, and thus decreases, or even prevents, oxidant-mediated damage. In addition, NAC may act as a direct scavenger for oxidants. Treatment with NAC provides beneficial effects in a number of respiratory, cardiovascular, endocrine, infectious, and other disease settings as described in WO05/017094, the contents of which are herein incorporated by reference. For example, rapid administration of NAC is the standard of care for preventing hepatic injury in acetaminophen overdose. NAC administered intravenously in dogs has been shown to protect against pulmonary oxygen toxicity and against ischemic and reperfusion damage [Gillissen, A., and Nowak, A., Respir. Med. 92: 609-23, 613 (1998)]. NAC treatment also has been shown to decrease NF-κB activation, which in turn decreases neutrophilic inflammation in the lung.

### Antioxidant Therapy in Chronic Pulmonary Diseases

The lung exists in a high-oxygen environment, and together with its large surface area and blood supply, is highly susceptible to injury mediated by oxidative stress. Since reactive oxygen species are constantly formed in the lung, and since oxygen metabolites are believed to play a predominant role in the pathogenesis of various pulmonary inflammatory disorders, antioxidant therapy would seem to be a rational approach to take in pulmonary diseases. Patients with acute respiratory distress syndrome (ARDS), idiopathic pulmonary fibrosis (IPF), or chronic obstructive pulmonary disorder (COPD) have been the primary targets for clinical studies evaluating the efficacy of NAC in antioxidant therapy. The results have been, for the most part, inconclusive.

COPD, a syndrome of chronic airway inflammation, initiated in most cases by chronic tobacco smoke exposure, which damages the airways and lung parenchyma over many years, has been extensively studied in this regard. An accelerated functional deterioration is accompanied by the development of cough, sputum production, dyspnea, and abnormal gas exchange, and leads to an increasing risk of acute flares of disease referred to as exacerbations. Exacerbation frequency increases as the disease progresses, further accelerating lung function decline.

The presence of oxidative stress in the airways of smokers and patients with COPD has been shown by increased products of lipid peroxidation and altered antioxidant status. Patients with COPD are known to have increased numbers of activated neutrophils in their airways that are believed to be attracted to the airways by the cytokines IL-8 and TNF-α, which are present in increased levels in the lungs of patients with stable COPD. Drost, E. M., Skwarski, K. N., Sauleda, J., Soler, N., Roca, J., Agusti, A., MacNee, W. “Oxidative Stress and Airway Inflammation in Severe Exacerbations of COPD,” Thorax 60: 293-300 (2005) disclose that exacerbations of COPD are considered to reflect worsening of the underlying chronic inflammation in the airways. They reported that increased oxidative stress in the airways of patients with COPD is increased further in severe and very severe exacerbations of the disease and is associated with increased neutrophil influx and levels of IL-8, an inflammatory cytokine associated with airway inflammation in COPD. The study acknowledged that in COPD, the interpretation of differences between exacerbations and the stable state may actually be a reflection of differences in disease severity, because exacerbations were studied in patients with severe and very severe underlying COPD and compared with stable patients with moderate disease.

While there is some evidence that oral NAC offsets chronic redox stress when administered in the long term for chronic respiratory conditions, some studies have demonstrated a beneficial effect, but others have not. For example, NAC has been used for over 20 years to treat COPD, a disease not characterized by glutathione deficiency. Gillissen and Nowak, Respir. Med. 92: 609-23, 615 (1998), for example, reported that improvements in glutathione levels were seen in patients with ARDS and IPF, but not COPD, who received 600-1800 mg NAC given daily by mouth. Oral NAC at high doses (generally 1.2 to 1.8 g/day) has been proposed for the treatment (preventive or symptomatic) of exacerbations in a subset of patients with COPD who are not receiving inhaled corticosteroids (Sutherland, E. R., et al., COPD Chronic Obstructive Pulmonary Disease 3: 195-202 (2006)). Although treatment with 600 mg oral NAC per day was ineffective at preventing deterioration in lung function and exacerbations in patients with COPD who had frequent exacerbations (i.e., at least two per year for 2 years), these investigators suggested that higher doses of NAC, such as 1200 mg or 1800 mg per day, could be assessed in future trials (Decramer, M., Lancet 365: 1552-60 (2005)). Oral NAC at high doses (generally 1.2 to 1.8 g/day) also has been proposed for the treatment (preventive or symptomatic) of exacerbations in chronic bronchitis, an inflammation, or irritation, of the airways in the lungs characterized by a chronic cough and chronic mucus production without another known cause (see Grandjean, E. M. et al., Clinical Therapeutics 22(2): 209-21 (2000), and Stey, C., et al., Eur. Resp. J. 16: 253-62 (2000)).

### Cystic Fibrosis

Cystic fibrosis (CF) is an inherited autosomal recessive disorder. It is one of the most common fatal genetic disorders in the United States, affecting about 30,000 individuals, and is most prevalent in the Caucasian population, occurring in one of every 3,300 live births. The gene involved in cystic fibrosis, which was identified in 1989, codes for a protein called the cystic fibrosis transmembrane conductance regulator (CFTR). CFTR is normally expressed by exocrine epithelia throughout the body and regulates the movement of chloride ions, bicarbonate ions and glutathione into and out of cells. In cystic fibrosis patients, mutations in the CFTR gene lead to alterations or total loss of CFTR protein function, resulting in defects in osmolarity, pH and redox properties of exocrine secretions. In the lungs, CF manifests itself by the presence of a thick mucus secretion which clogs the airways. In other exocrine organs, such as the sweat glands, CF may not manifest itself by an obstructive phenotype, but rather by abnormal salt composition of the secretions (hence the clinical sweat osmolarity test to detect CF patients).

The predominant cause of illness and death in cystic fibrosis patients is progressive lung disease. The thickness of CF mucus, which blocks the airway passages, is believed to stem from abnormalities in osmolarity of secretions, as well as from the presence of massive amounts of DNA, actin, proteases and prooxidative enzymes originating from a subset of inflammatory cells, called neutrophils. Indeed, CF lung disease is characterized by early, hyperactive neutrophil-mediated inflammatory reactions to both viral and bacterial pathogens.

The hyperinflammatory syndrome of CF lungs has several underpinnings, among which an imbalance between pro-inflammatory chemokines, chiefly IL-8, and anti-inflammatory cytokines, chiefly IL-10, seems to play a major role. See Chmiel et al. Clin Rev Allergy Immunol. 3(1):5-27 (2002). Chronic oxidative stress in CF patients may severely affect the deformability of blood neutrophils circulating in CF lung capillaries, thereby increasing their recruitment to the lungs. See Hogg. Physiol Rev. 67(4):1249-95 (1987). Chronic oxidative stress in CF is linked to the overwhelming release of oxidants by inflammatory lung neutrophils and to abnormal antioxidant defenses caused by malabsorption of dietary antioxidants through the gut and a possible defect in GSH efflux. See Wood et al. J. Am. Coll. Nutr. 20(2 Suppl):157-165 (2001).

The hyperinflammatory syndrome at play in CF lungs may predispose such patients to chronic infections with opportunistic bacterial pathogens. The most common bacterium to infect the CF lung is Pseudomonas aeruginosa, a gram-negative microorganism. The lungs of most children with CF become colonized by P. aeruginosa before their third birthday. By their tenth birthday, P. aeruginosa becomes dominant over other opportunistic pathogens. See Gibson et al., Am. J. Respir. Crit. Care Med., 168(8): 918-951 (2003). P. aeruginosa infections further exacerbate neutrophilic inflammation, which causes repeated episodes of intense breathing problems in CF patients. Although antibiotics may decrease the frequency and duration of these attacks, the bacterium progressively establishes a permanent residence in CF lungs by switching to a so-called “mucoid”, biofilm form of high resistance and low virulence, which never may be eliminated completely from the lungs. The continuous presence in CF lungs of inflammatory by-products, such as extracellular DNA and elastase, could play a major role in selecting for mucoid P. aeruginosa forms. See Walker et al. Infect Immun. 73(6): 3693-3701 (2005).

Treatments for CF lung disease typically involve antibiotics, anti-inflammatory drugs, bronchodilators, and chest physiotherapy to help fight infection, neutrophilic inflammation and obstruction and clear the airways. Nevertheless, the persistent, viscous and toxic nature of airway secretions in cystic fibrosis lung disease still leads to progressive deterioration of lung function. See Rancourt et al., Am. J. Physiol. Lung Cell Mol. Physiol. 286(5): L931-38 (2004).

Although it is characterized by heavy inflammation, CF historically was thought to be a mucus disease. N-acetylcysteine (NAC) is a widely used mucolytic drug in patients with a variety of disorders, including cystic fibrosis. See Rochat, et al., J. Cell Physiol. 201(1): 106-16 (2004). It has been hypothesized that NAC works as a mucolytic by rupturing the disulfide bridges of the high molecular weight glycoproteins present in the mucus, resulting in smaller subunits of the glycoproteins and reduced mucous viscosity. Id. To this end, researchers and clinicians have administered NAC to CF patients generally by nebulization, as well as orally. Two placebo-controlled studies have reported beneficial effects of oral NAC treatment on lung function in cystic fibrosis. See G. Stafanger, et al., Eur. Respir. J. 1(2): 161-67 (1988). Active treatment consisted of NAC administered as a 200 mg oral dose three times daily (for patients weighing less than 30 kg) or as a 400 mg oral dose two times daily (for patients weighing more than 30 kg). Ratjen, F., et al., Eur. J. Pediatr. 144(4): 374-78 (1985) reported improvement in some measures of lung function but saw no significant clinical differences between patients treated with oral NAC (200 mg 3 times a day), the secretolytic drug ambroxol (30 mg, three times daily), and placebo. A very short fourth study (2 weeks) failed to find any significant difference between the trial arms. See Gotz et al, Eur. J. Resp. Dis. 61 (Suppl) 111: 122-26 (1980).

Duijvestijn, Y. C. and Brand, P. L. Acta Paediatr. 88(1): 38-41 (1999) observed, however, that despite the fact that NAC commonly is used in CF, there is remarkably little published data on its effects. They tested their hypothesis that NAC's antioxidant properties could be useful in preventing decline of lung function (defined as forced expiratory volume in one second, or FEV1) in cystic fibrosis by performing a systematic review of the literature to evaluate whether published evidence supports the use of NAC administered orally or by nebulization to improve lung function in patients with cystic fibrosis. They identified 23 papers, the majority of which were uncontrolled clinical observations, of which only three randomized controlled trials on nebulized NAC were found. None of these studies showed a statistically significant or clinically relevant beneficial effect of NAC aerosol. They found a small beneficial effect of doubtful clinical relevance of oral NAC on FEV1 in CF. Although they suggested that the effects of long-term treatment with oral NAC on lung function in CF should be investigated, they concluded that there is no evidence supporting the use of N-acetylcysteine in cystic fibrosis.

Despite these findings, redox-based therapy is an attractive idea for CF, since redox imbalance is a well-recognized aspect of the disease, yet seldom considered as a therapeutic target. See Cantin, Curr Opin Pulm Med. 10(6):531-6 (2004). Systemic oxidative stress may affect blood neutrophils by lowering their intracellular GSH levels, which in turn renders them more prone to lung trapping and dysfunction. See Hogg. Physiol Rev. 67(4):1249-95 (1987). Besides, systemic oxidative stress may alter the chemokine/cytokine balance, favoring inflammation, which systemic NAC treatment may help alleviate. See Zafarullah et al. Cell Mol Life Sci. 60(1):6-20 (2003).

U.S. application Ser. No. 11/507,706, the contents of which are expressly incorporated herein by reference, describes an investigation into whether NAC in high doses could counter systemic oxidative stress/redox imbalance and inhibit inflammation when administered orally to CF patients. Blood neutrophils were targeted before they reach the lung, a strategy that had not been tested in CF. The inflammatory and redox aspects of CF lung disease, which are major contributors to the progression of the disease, were the focus of that study.

### Acute Exacerbations of Pulmonary Disease

A systematic review of randomized controlled trials for established acute oxidative/inflammatory syndromes, such as Acute Respiratory Distress Syndrome (ARDS), which is characterized by diffuse inflammation of the lung's alveolar-capillary membrane in response to various pulmonary and extrapulmonary insults, and Acute Lung Injury (ALI), a milder form of lung injury, showed that NAC had no effect on early mortality in these diseases (Adhikari, N., Burns, K E A, Meade, M O, The Cochrane Library 1:1-43, John Wiley & Sons, Ltd., 2008).

Acute exacerbations of CF are characterized by increased oxidative stress and sputum concentrations of bioactive lipid mediators. Reid, D. W., et al., Respirology 12 (1): 63-69 (2007). McGrath, L. T. et al, “Oxidative stress during acute respiratory exacerbations in cystic fibrosis,” Thorax 54: 518-523 (1999) have reported that during acute respiratory exacerbations, patients with CF are subject to acute oxidative attack in addition to the chronic systemic oxidative stress found in this condition. Such acute respiratory exacerbations in CF are characterized by increased respiratory symptoms, reduction in forced expiratory volume in one second (“FEV1”) of more than 10%, and a decision to treat with intravenous antibiotics. As reported, although almost all of the antioxidant scavengers developed to cope with the acute attack were partially depleted during infection, antibiotic treatment of the acute infection tended to reduce measures of free radical damage by moderating the infection and hence the immune response.

Like in CF, it is known that chronic phase and acute pathological flares of such chronic pulmonary diseases as Acute Respiratory Distress Syndrome (ARDS), Acute Lung Injury (ALI), Chronic Bronchitis (CB), and Chronic Obstructive Pulmonary Disease (COPD) share a common feature, i.e., their chronic phase and acute pathological flares are associated with redox and inflammatory dysfunctions and an increased proteolysis of lung tissue.

Unlike CF, ARDS, ALI, CB, and COPD, both Idiopathic Pulmonary Fibrosis (IPF) and Asthma are characterized by considerable matrix thickening/deposition in the mucosallumen of the airways. The effect of high-dose oral NAC has not been tested against acute exacerbations in either IPF or asthma.

Idiopathic Pulmonary Fibrosis (IPF), a syndrome regrouping several diseases with progressive fibrosis of the alveoli, is a chronic, progressive, incurable lung disease characterized by deposition of fibers in the lung through the hyperproliferation of myofibroblasts. Causative factors remain unknown. In some individuals, it develops quickly, while others have cryptic disease. An oxidant-antioxidant imbalance that depletes glutathione levels has been described in IPF.

A clinical study reported by Demedts, Maurits, et al., New England J. Med. 353 (21): 2229-42 (2005) has suggested that NAC may be beneficial when combined with standard therapies for chronic IPF, but the study was not powered to show the impact of NAC on survival, did not address use of NAC as a primary therapy in IPF patients, and did not address the effect of high-dose oral NAC on acute exacerbations of IPF. The double-blind, randomized, placebo-controlled multicenter study assessed the effectiveness over one year of 600 mg NAC administered three times daily added to standard therapy with prednisone plus azothioprine to test whether this regimen would slow the functional deterioration in patients with IPF has been reported. The primary endpoints were changes between baseline and month 12 in vital capacity (meaning the total amount of air that may be exhaled after a maximum inspiration) and in single-breath carbon monoxide diffusing capacity (“DL_{CO}”). The results of the study showed that NAC plus standard therapy (prednisone plus azothioprine) slows the deterioration of the primary endpoints vital capacity and DL_{CO} in patients with IPF better than does the standard therapy (prednisone plus azothioprine) alone.

Episodes of idiopathic acute respiratory deterioration have been termed acute exacerbations of IPF. Collard, H. R. et al., Am. J. Respir. Crit. Care med. 176(7): 636-43 (2007). The etiology of acute exacerbations of IPF is unknown. There are several competing hypotheses, including, but not limited to, the hypothesis that acute exacerbations of IPF represents a distinct, pathobiological manifestation of the primary disease process, characterized by idiopathic lung injury; the hypothesis that acute exacerbations of IPF may represent clinically occult but biologically distinct conditions that go undiagnosed, such as viral infection, or aspiration; and the hypothesis that acute exacerbations of IPF may be the sequelae of an acute direct stress to the lung, with a subsequent acceleration of the already abnormal fibroproliferative process intrinsic to IPF.

Asthma is an inflammatory disease of the lungs characterized by reversible (in most cases) airway obstruction due to narrowing of the conducting airways, hyper-responsiveness/hyper-reactivity, and chronic inflammation characterized by an influx and activation of inflammatory cells, generation of inflammatory mediators, and epithelial cell shedding. In chronic asthma, there is an increased sequestration within the lungs of leukocytes from the peripheral microcirculation. Since many chronic asthma patients have eosinophilic infiltrates, eosinophils are thought to play a critical role in the inflammatory response in chronic asthma. Indeed, it is believed that much of the lung problems in chronic asthma relates to the eosinophil disease. In addition, neutrophils isolated from peripheral blood of asthmatic patients generate greater amounts of reactive oxygen species than cells from normal subjects, may be involved in acute exacerbations of asthma. (Kirkham, P., Rahman, I., Pharamacology & Therapeutics 111: 476-94 (2006)).

Oxidative stress is believed to play a key role in the pathogenesis of clinically stable (chronic) bronchial asthma. It also has been shown that acute exacerbations of asthma [meaning a sudden increase in breathlessness over the preceding 48 hours and presence of one of the following signs: tachypnea (meaning a respiratory rate of >18), use of accessory muscles or respiration, audible wheezing, prolonged expiration with rhonchi (meaning a sound occurring during inspiration or expiration caused by air passing through bronchi that are narrowed by inflammation, spasm of smooth muscle, or presence of mucus in the lumen heard on auscultation (meaning a diagnostic method of listening to the sounds made) of the chest] are associated with increased inflammation in the airways and with increased oxidative stress. Nadeem, A., et al., J. Asthma 1:45-50 (2005).

Asthmatic exacerbations commonly occur in two phases: an immediate phase, caused by release of mediators, that often is characterized by bronchoconstriction resulting in wheezing and coughing, and an inflammatory or late phase, that includes increasing airway inflammation, which leads to hyper-responsiveness.

There are many published guidelines for management of asthma available, but there is little if any documented objective data to support their usefulness in acute care of asthma.

Although chronic redox and inflammatory stresses in asthma (Nadeem, 2005; Kirkham 2006) have been documented, the effect of high-dose oral NAC has not been tested against acute exacerbations in asthma.

Tuberculosis (TB), once believed to have been almost eradicated, has shown a resurgence and a substantial increase in drug resistance. Human immunodeficiency virus (HIV) infection is a major risk factor for the development of TB, and TB seems to make HIV infection worse [Sacchetini, J. C., et al. Nat. Rev. Microbiol. 6(1):41-52 (2008)]. Immune reconstitution inflammatory syndrome (referred to herein as IRS or IRIS), is an adverse consequence of the restoration of pathogen-specific immune responses in HIV infected patients during the initial months of highly active anti-retroviral therapy. Symptoms include fever, lymphadenopathy, and worsening of respiratory and other TB symptoms Although the pathophysiology of IRIS is unknown, preliminary investigations suggest that an acute exacerbation of mycobacterium-specific Th1 responses against mycobacterial antigens after HIV infection control by this therapy may cause IRIS in HIV/TB patients. See Bougarit, A. et al., AIDS 20: F1-F7 (2006); Shankar, E. M., AIDS Research & Therapy 4: 29 (2007).

The present invention describes use of NAC as a primary therapy for acute exacerbations of CF, IPF, asthma and TB.

### SUMMARY OF THE INVENTION

The present invention describes compositions and methods for treating acute exacerbations of an inflammatory lung disease. In one aspect, the present invention provides a method of treating the symptoms of an acute exacerbation of an inflammatory lung disease other than COPD in a patient in need thereof, the method comprising the step of: (a) administering to a patient in need thereof a pharmaceutical composition comprising (1) an acute exacerbation-reducing amount of N-acetylcysteine, a pharmaceutically acceptable salt of N-acetylcysteine, or a pharmaceutically acceptable derivative of N-acetylcysteine, and (2) a pharmaceutically acceptable carrier, and thereby modulating at least one symptom of the acute exacerbation. According to one embodiment of the method, the inflammatory lung disease is cystic fibrosis. According to another embodiment, the inflammatory lung disease is an interstitial lung disease. According to another embodiment, the interstitial lung disease is idiopathic pulmonary fibrosis. According to another embodiment, the inflammatory lung disease is asthma. According to another embodiment, the inflammatory lung disease is tuberculosis and the patient is an HIV patient. According to another embodiment, ding to claim 1, wherein in step (a) of the method the pharmaceutical composition is administered systemically by a route selected from the group consisting of orally, buccally, topically, by inhalation, by insufflation, parenterally and rectally. According to another embodiment, in step (a) of the method, the pharmaceutical composition is administered orally. According to another embodiment, the acute exacerbation-reducing amount of N-acetylcysteine, a pharmaceutically acceptable salt of N-acetylcysteine, or a pharmaceutically acceptable derivative of N-acetylcysteine in the pharmaceutical composition administered orally is about 1.8 grams per day to about 6 grams per day, and less than or equal to 200 mg per kg per day. According to another embodiment, the acute exacerbation-reducing amount of N-acetylcysteine, a pharmaceutically acceptable salt of N-acetylcysteine, or a pharmaceutically acceptable derivative of N-acetylcysteine in the pharmaceutical composition administered orally is at least about 1800 mg per day and less than or equal to 200 mg per kg per day. According to another embodiment, the acute exacerbation-reducing amount of N-acetylcysteine, a pharmaceutically acceptable salt of N-acetylcysteine, or a pharmaceutically acceptable derivative of N-acetylcysteine in the pharmaceutical composition administered orally is at least about 2400 mg per day and less than or equal to 200 mg per kg per day. According to another embodiment, the acute exacerbation-reducing amount of N-acetylcysteine, a pharmaceutically acceptable salt of N-acetylcysteine, or a pharmaceutically acceptable derivative of N-acetylcysteine in the pharmaceutical composition administered orally is at least about 3000 mg per day and less than or equal to 200 mg per kg per day. According to another embodiment, in step (a) of the method, the pharmaceutical composition is administered parenterally. According to another embodiment, the acute exacerbation-reducing amount of N-acetylcysteine, a pharmaceutically acceptable salt of N-acetylcysteine, or a pharmaceutically acceptable derivative of N-acetylcysteine in the pharmaceutical composition administered parenterally is about 200 mg NAC to about 2000 mg NAC per dosage unit. According to another embodiment, the method further comprises the step of (b) administering a pharmaceutically effective amount of a disease-specific therapeutic agent. According to another embodiment, the disease specific therapeutic agent comprises at least one cystic fibrosis therapeutic agent selected from the group consisting of an anti-infective agent, a bronchodilating agent, and an anti-inflammatory agent. According to another embodiment, the disease-specific therapeutic agent comprises at least one idiopathic pulmonary fibrosis therapeutic agent selected from the group consisting of a corticosteroid agent, an anticoagulation agent, pirfenidone, and an antimicrobial agent. According to another embodiment, the disease-specific therapeutic agent comprises at least one asthma therapeutic agent selected from the group consisting of an antimicrobial agent, a bronchodilator agent, a corticosteroid; a leukotriene antagonist; and a α-agonist. According to another embodiment, the disease specific therapeutic agent comprises at least one tuberculosis therapeutic agent. According to another embodiment, the cystic fibrosis therapeutic agent is at least one agent selected from the group consisting of an anti-infective agent, a bronchodilating agent, and an anti-inflammatory agent. According to another embodiment, the method further comprising the step of (b) administering a respiratory therapy to the patient. According to another embodiment, the method further comprising the step of (b) administering a rehabilitation therapy to the patient.

In another aspect, the present invention provides a pharmaceutical kit for treating an acute exacerbation of an inflammatory lung disease other than COPD in a subject in need thereof, the kit comprising a) a first container containing a pharmaceutically effective amount of a disease-specific therapeutic agent, and b) a second container containing a pharmaceutical composition comprising (i) an acute exacerbation-reducing amount of N-acetylcysteine, a pharmaceutically acceptable salt of N-acetylcysteine, or a pharmaceutically acceptable derivative of N-acetylcysteine, and (ii) a pharmaceutically acceptable carrier. According to one embodiment, the disease specific agent in the first container comprises at least one cystic fibrosis agent selected from the group consisting of an anti-infective agent, a bronchodilating agent, and an anti-inflammatory agent. According to another embodiment, the disease-specific agent in the first container comprises at least one idiopathic pulmonary fibrosis therapeutic agent selected from the group consisting of a corticosteroid agent, an anticoagulation agent, pirfenidone, and an antimicrobial agent. According to another embodiment, the disease-specific agent in the first container comprises at least one asthma therapeutic agent selected from the group consisting of an antimicrobial agent, a bronchodilator agent, a corticosteroid; a leukotriene antagonist; and a 0-agonist. According to another embodiment, the disease specific agent comprises at least one tuberculosis therapeutic agent.

### DETAILED DESCRIPTION OF THE INVENTION

The present invention describes compositions and methods for treating acute exacerbations of an inflammatory lung disease. In some embodiments, the inflammatory lung disease is bronchial asthma. In some embodiments, the inflammatory lung disease is Idiopathic Pulmonary Fibrosis (IPF). In some embodiments, the inflammatory lung disease is cystic fibrosis. In some embodiments, the inflammatory lung disease is tuberculosis, with or without co-infection with HIV.

The term “acute” as used herein refers to a rapid onset, brief (not prolonged), and severe health-related state.

The term “chronic” refers to a persistent, long-term, health-related state of 3 months duration or longer.

The term “condition,” as used herein, refers to a variety of health states and is meant to include disorders or diseases, and inflammation caused by any underlying mechanism or disorder.

The term “disease” or “disorder,” as used herein, refers to an impairment of health or a condition of abnormal functioning.

The term “exacerbations” as used herein refers to an increase in the severity of a disease or any of its signs or symptoms.

The term “idiopathic” refers to a disease of unknown cause.

The term interstitial lung disease (“ILD”) includes a variety of chronic lung disorders in which lung tissue is damaged in some known or unknown way, the walls of the air sacs in the lung become inflamed; and scarring (or fibrosis) begins in the interstitium (or tissue between the air sacs) and the lung becomes stiff. When all known causes of interstitial lung disease have been ruled out, the condition is called idiopathic pulmonary fibrosis.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

As used herein, the term “modulate” or “modulating” refers to adjusting, changing, or manipulating the function or status of at least one of redox balance or inflammation in cystic fibrosis. Such modulation may be any change, including an undetectable change. In one embodiment of the present invention, a method of treating an inflammation in cystic fibrosis patients comprises the steps of administering to a patient in need thereof a composition comprising an inflammation-reducing amount of NAC, a pharmaceutically acceptable salt of NAC, or a pharmaceutically acceptable derivative of NAC, and a pharmaceutically acceptable carrier and a pharmaceutically acceptable carrier, thereby modulating the inflammation.

Intracellular redox status plays a critical role in cell function. The term “oxidative stress” as used herein refers to a condition caused by an imbalance between reactive oxygen species and the antioxidant defense mechanisms of a cell, leading to an excess production of oxygen metabolites. Skaper, et al., Free Radical Biol. & Med. 22(4): 669-678 (1997).

The term “redox imbalance” as used herein refers to the imbalance between reactive oxygen species and the antioxidant defense mechanisms of a cell.

The term “syndrome,” as used herein, refers to a pattern of symptoms indicative of some disease or condition.

As used herein the term “treating” includes abrogating, substantially inhibiting, slowing or reversing the progression of a condition, substantially ameliorating clinical or symptoms of a condition, and substantially preventing the appearance of clinical or symptoms of a condition.

In one embodiment of the present invention, the composition of the present invention comprises an inflammation-reducing amount of NAC and a pharmaceutically acceptable carrier. In another embodiment of the present invention, the composition of the present invention comprises a redox imbalance adjusting amount of NAC and a pharmaceutically acceptable carrier. In another embodiment of the present invention, the composition of the present invention comprises an acute exacerbation-reducing amount of NAC and a pharmaceutically acceptable carrier.

As used herein the terms “inflammation-reducing amount,” “redox imbalance adjusting amount”, “acute exacerbation-reducing amount,” or “pharmaceutically effective amount” refer to the amount of the compositions of the invention that result in a therapeutic or beneficial effect following its administration to a subject. The inflammation-reducing, redox imbalance adjusting, acute exacerbation-reducing, or pharmaceutically effective amount may be curing, minimizing, preventing or ameliorating a disease or disorder, or may have any other anti-inflammatory, redox balancing or pharmaceutical beneficial effect. Without being limited by theory, it is believed that an acute exacerbation reducing amount of NAC may be an amount that may increase a threshold for acute pathways of inflammation; that may act on a new pathway that acts on a T-cell subset that controls neutrophil infiltration in the lung; and/or that may act on signaling pathways inside other cells and inhibit ability of neutrophils to enter the lung. The concentration of the substance is selected so as to exert its inflammation-reducing, redox balancing, or pharmaceutical effect, but low enough to avoid significant side effects within the scope and sound judgment of the skilled artisan. The effective amount of the composition may vary with the age and physical condition of the biological subject being treated, the severity of the condition, the duration of the treatment, the nature of concurrent therapy, the specific compound, composition or other active ingredient employed, the particular carrier utilized, and like factors.

A skilled artisan may determine a pharmaceutically effective amount of the inventive compositions by determining the unit dose. As used herein, a “unit dose” refers to the amount of inventive composition required to produce a response of 50% of maximal effect (i.e. ED50). The unit dose may be assessed by extrapolating from dose-response curves derived from in vitro or animal model test systems. The amount of compounds in the compositions of the present invention which will be effective in the treatment of a particular disorder or condition will depend on the nature of the disorder or condition, and may be determined by standard clinical techniques. (See, for example, Goodman and Gilman's THE PHARMACOLOGICAL BASIS OF THERAPEUTICS, Joel G. Harman, Lee E. Limbird, Eds.; McGraw Hill, New York, 2001; THE PHYSICIAN'S DESK REFERENCE, Medical Economics Company, Inc., Oradell, N.J., 1995; and DRUG FACTS AND COMPARISONS, FACTS AND COMPARISONS, INC., St. Louis, Mo., 1993). The precise dose to be employed in the formulation will also depend on the route of administration, and the seriousness of the disease or disorder, and should be decided according to the judgment of the practitioner and each patient's circumstances.

The term “pharmaceutical composition,” as used herein, refers to a composition that has under gone federal regulatory review, which prevents, reduces in intensity, cures, ameliorates, or otherwise treats a target disorder or disease. It is preferred that the pharmaceutical compositions according to the present invention contain from about at least 200 to about 2000 mg NAC per dosage unit for oral administration and about at least 200 to about 2000 mg NAC per dosage unit for parenteral administration at the physician's discretion. Usual dosage should be between 1.8 to 6.0 g/d, not to exceed 200 mg/kg/d.

The unit dose of NAC, will usually comprise at least about 200 mg (for pediatric doses), usually at least about 600 mg (for adult doses); and usually not more than about 2000 mg at the physician's discretion, from a minimum of one to a maximum of six daily intakes. Patients on therapy known to deplete cysteine/glutathione or produce oxidative stress may benefit from higher amounts of NAC.

The terms “drug carrier”, “carrier”, or “vehicle” as used herein refers to a pharmaceutically acceptable inert agent or vehicle for delivering one or more active agents to a mammal, and often is referred to as “excipient.” As used herein the term “a pharmaceutically acceptable carrier” refers to any substantially non-toxic carrier conventionally useable for NAC administration in which NAC will remain stable and bioavailable. The carrier suitable for NAC administration must be of sufficiently high purity and of sufficiently low toxicity to render it suitable for administration to the mammal being treated. Carriers and vehicles useful herein include any such materials known in the art which are nontoxic and do not interact with other components. The (pharmaceutical) carrier may be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulphate, etc.). Other suitable (pharmaceutical) carriers for the compositions of the present invention include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like.

In some embodiments, the carrier of the composition of the present invention includes a release agent such as sustained release or delayed release carrier. In such embodiments, the carrier may be any material capable of sustained or delayed release to provide a more efficient administration, e.g., resulting in less frequent and/or decreased dosage, improve ease of handling, and extend or delay effects on diseases, disorders, conditions, syndromes, and the like, being treated. Non-limiting examples of such carriers include liposomes, microsponges, microspheres, or microcapsules of natural and synthetic polymers and the like. Liposomes may be formed from a variety of phospholipids such as cholesterol, stearylamines or phosphatidylcholines.

It is preferred that the NAC be substantially free of sulfones or other chemicals that interfere with the metabolism of any co-administered drug in its bioactive form. It is also preferred that the NAC be substantially free of its oxidized form, di-N-acetylcysteine and that the composition should be prepared in a manner that substantially prevents oxidation of the NAC during preparation or storage.

It may be noted that the effectiveness of NAC depends on the presence of the reduced form, which may, for example, liberate the reduced form of glutathione from homo- and hetero-disulfide derivatives in thiol-disulfide exchange reactions. A typical unit dosage may be a solution suitable for oral or intravenous administration; an effervescent tablet suitable for dissolving in water, fruit juice, or carbonated beverage and administered orally; a tablet taken from two to six times daily, or one time-release capsule or tablet taken several times a day and containing a proportionally higher content of active ingredient, etc. The time-release effect may be obtained by capsule materials that dissolve at different pH values, by capsules that release slowly by osmotic pressure, or by any other known means of controlled release. Unit dosage forms may be provided wherein each dosage unit, for example, teaspoonful, tablespoonful, gel capsule, tablet or suppository, contains a predetermined amount of the compositions of the present invention. Similarly, unit dosage forms for injection or intravenous administration may comprise the compound of the present invention in a composition as a solution in sterile water, normal saline or another pharmaceutically acceptable carrier. The specifications for the unit dosage forms of the present invention depend on the effect to be achieved and the intended recipient. Thus, in some embodiments, NAC is formulated at high doses as an effervescent tablet or in granular form in a single dose packet to be dissolved in water to prevent untoward stomach effects.

Over-the-counter NAC may be variably produced and packaged. Because the production and packaging methods generally do not guard against oxidation, the NAC may be significantly contaminated with bioactive oxidation products. These may be particularly important in view of data indicating that the oxidized form of NAC has effects counter to those reported for NAC and is bioactive at doses roughly 10-100 fold less than NAC. See Sarnstrand et al J. Pharmacol. Exp. Ther. 288:1174-84 (1999).

The distribution of the oxidation states of NAC as a thiol and disulfide depends on the oxidation/reduction (redox) potential. The half-cell potential obtained for the NAC thiol/disulfide pair is about +63 mV, indicative of its strong reducing activity among natural compounds [see Noszal et al. J. Med. Chem. 43:2176-2182 (2000)]. In a preferred embodiment of the invention, the preparation and storage of the formulation is performed in such a way that the reduced form of NAC is the primary form administered to the patient. Maintaining NAC containing formulations in solid form is preferable for this purpose. When in solution, NAC containing formulations are preferably stored in a brown bottle that is vacuum sealed. Storage in cool dark environments is also preferred.

The determination of reduced and oxidized species present in a sample may be determined by various methods known in the art, including, but not limited to, for example, capillary electrophoresis, and high performance liquid chromatography as described by Chassaing et al. J. Chromatogr. B. Biomed. Sci. Appl. 735(2):219-27 (1999).

The compositions of the present invention may be administered systemically either orally, parenterally, or rectally in dosage unit formulations containing conventional nontoxic pharmaceutically acceptable carriers, adjuvants, and vehicles as desired.

The compositions of the present invention may be in a form suitable for oral use, for example, as tablets, troches, lozenges, aqueous or oily suspensions, dispersible powders or granules, emulsions, hard or soft capsules or syrups or elixirs. Compositions intended for oral use may be prepared according to any known method, and such compositions may contain one or more agents selected from the group consisting of sweetening agents, flavoring agents, coloring agents, and preserving agents in order to provide pharmaceutically elegant and palatable preparations. Tablets may contain the active ingredient(s) in admixture with non-toxic pharmaceutically-acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They also may be coated for controlled release.

Compositions of the present invention also may be formulated for oral use as hard gelatin capsules, where the active ingredient(s) is(are) mixed with an inert solid diluent, for example, calcium carbonate, calcium phosphate or kaolin, or soft gelatin capsules wherein the active ingredient(s) is (are) mixed with water or an oil medium, for example, peanut oil, liquid paraffin, or olive oil.

The compositions of the present invention may be formulated as aqueous suspensions wherein the active ingredient(s) is (are) in admixture with excipients suitable for the manufacture of aqueous suspensions. Such excipients are suspending agents, for example, sodium carboxymethylcellulose, methylcellulose, hydroxy-propylmethylcellulose, sodium alginate, polyvinylpyrrolidone, gum tragacanth, and gum acacia; dispersing or wetting agents may be a naturally-occurring phosphatide such as lecithin, or condensation products of an alkylene oxide with fatty acids, for example, polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions also may contain one or more coloring agents, one or more flavoring agents, and one or more sweetening agents, such as sucrose or saccharin.

Compositions of the present invention may be formulated as oily suspensions by suspending the active ingredient in a vegetable oil, for example arachis oil, olive oil, sesame oil or coconut oil, or in a mineral oil, such as liquid paraffin. The oily suspensions may contain a thickening agent, for example, beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Compositions of the present invention may be formulated in the form of dispersible powders and granules suitable for preparation of an aqueous suspension by the addition of water. The active ingredient in such powders and granules is provided in admixture with a dispersing or wetting agent, suspending agent, and one or more preservatives. Suitable dispersing or wetting agents and suspending agents are exemplified by those already mentioned above. Additional excipients, for example, sweetening, flavoring and coloring agents also may be present.

Compositions of the invention also may be formulated as a beverage or as an additive to a beverage, where the term “beverage” refers to any non-alcoholic flavored carbonated drink, soda water, non-alcoholic still drinks, diluted fruit or vegetable juices whether sweetened or unsweetened, seasoned or unseasoned with salt or spice, or still or carbonated mineral waters used as a drink. The term “additive” as used herein refers to any substance the intended use of which results, or may reasonably be expected to result, directly or indirectly, in its becoming a component or otherwise affecting the characteristics of any beverage. In some embodiments, the beverage is a flavored carbonated beverage. In some embodiments, the beverage is a flavored non-carbonated beverage. In some embodiments, the beverage is a natural fruit beverage. The beverage also may contain one or more coloring agents, one or more flavoring agents, one or more sweetening agents, one or more antioxidant agents, and one or more preservatives.

Compositions of the invention also may be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate. The emulsions also may contain sweetening and flavoring agents.

Compositions of the invention also may be formulated as syrups and elixirs. Syrups and elixirs may be formulated with sweetening agents, for example, glycerol, propylene glycol, sorbitol or sucrose. Such formulations also may contain a demulcent, a preservative, and flavoring and coloring agents. Demulcents are protective agents employed primarily to alleviate irritation, particularly mucous membranes or abraded tissues. A number of chemical substances possess demulcent properties. These substances include the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

The compositions of the present invention may be in the form of a sterile injectable aqueous or oleaginous suspension. The term “parenteral” as used herein includes subcutaneous injections, intravenous, intramuscular, intrasternal injection, or infusion techniques. Injectable preparations, such as sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran. Optionally, the suspension may also contain stabilizers.

The term “topical” refers to administration of an inventive composition at, or immediately beneath, the point of application. The phrase “topically applying” describes application onto one or more surfaces(s) including epithelial surfaces. Although topical administration, in contrast to transdermal administration, generally provides a local rather than a systemic effect, as used herein, unless otherwise stated or implied, the terms topical administration and transdermal administration are used interchangeably. For the purpose of this application, topical applications shall include mouthwashes and gargles.

Topical administration may also involve the use of transdermal administration such as transdermal patches or iontophoresis devices which are prepared according to techniques and procedures well known in the art. The terms “transdermal delivery system”, transdermal patch” or “patch” refer to an adhesive system placed on the skin to deliver a time released dose of a drug(s) by passage from the dosage form through the skin to be available for distribution via the systemic circulation. Transdermal patches are a well-accepted technology used to deliver a wide variety of pharmaceuticals, including, but not limited to, scopolamine for motion sickness, nitroglycerin for treatment of angina pectoris, clonidine for hypertension, estradiol for post-menopausal indications, and nicotine for smoking cessation.

Patches suitable for use in the present invention include, but are not limited to, (1) the matrix patch; (2) the reservoir patch; (3) the multi-laminate drug-in-adhesive patch; and (4) the monolithic drug-in-adhesive patch; TRANSDERMAL AND TOPICAL DRUG DELIVERY SYSTEMS, pp. 249-297 (Tapash K. Ghosh et al. eds., 1997), hereby incorporated herein by reference. These patches are well known in the art and generally available commercially.

The compositions of the present invention may be in the form of a dispersible dry powder for pulmonary delivery. Dry powder compositions may be prepared by processes known in the art, such as lyophilization and jet milling, as disclosed in International Patent Publication No. WO 91/16038 and as disclosed in U.S. Pat. No. 6,921,527, the disclosures of which are incorporated by reference. The composition of the present invention is placed within a suitable dosage receptacle in an amount sufficient to provide a subject with a unit dosage treatment. The dosage receptacle is one that fits within a suitable inhalation device to allow for the aerosolization of the dry powder composition by dispersion into a gas stream to form an aerosol and then capturing the aerosol so produced in a chamber having a mouthpiece attached for subsequent inhalation by a subject in need of treatment. Such a dosage receptacle includes any container enclosing the composition known in the art such as gelatin or plastic capsules with a removable portion that allows a stream of gas (e.g., air) to be directed into the container to disperse the dry powder composition. Such containers are exemplified by those shown in U.S. Pat. No. 4,227,522; U.S. Pat. No. 4,192,309; and U.S. Pat. No. 4,105,027. Suitable containers also include those used in conjunction with Glaxo's Ventolin® Rotohaler brand powder inhaler or Fison's Spinhaler® brand powder inhaler. Another suitable unit-dose container which provides a superior moisture barrier is formed from an aluminum foil plastic laminate. The pharmaceutical-based powder is filled by weight or by volume into the depression in the formable foil and hermetically sealed with a covering foil-plastic laminate. Such a container for use with a powder inhalation device is described in U.S. Pat. No. 4,778,054 and is used with Glaxo's Diskhaler® (U.S. Pat. Nos. 4,627,432; 4,811,731; and 5,035,237). All of these references are incorporated herein by reference.

The compositions of the present invention may be in the form of suppositories for rectal administration of the composition. These compositions may be prepared by mixing the drug with a suitable nonirritating excipient such as cocoa butter and polyethylene glycols which are solid at ordinary temperatures but liquid at the rectal temperature and will therefore melt in the rectum and release the drug. When formulated as a suppository the compositions of the invention may be formulated with traditional binders and carriers, such as triglycerides.

The therapeutically active agent of the present invention may be formulated per se or in salt form. The term “pharmaceutically acceptable salts” refers to nontoxic salts of NAC. Pharmaceutically acceptable salts include, but are not limited to, those formed with free amino groups such as those derived from hydrochloric, phosphoric, sulfuric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Additional compositions of the present invention may be readily prepared using technology which is known in the art such as described in Remington's Pharmaceutical Sciences, 18th or 19th editions, published by the Mack Publishing Company of Easton, Pa., which is incorporated herein by reference.

The present invention further provides a pharmaceutical pack or kit comprising one or more containers filled with one or more of the ingredients of the pharmaceutical compositions of the invention. Associated with such container(s) may be a notice in the form prescribed by a governmental agency regulating the manufacture, use or sale of pharmaceuticals or biological products, which notice reflects approval by the agency of manufacture, use or sale for human administration.

For example, in one embodiment, a pharmaceutical kit for treating inflammation in cystic fibrosis patients according to the present invention includes a first container filled with a pharmaceutically effective amount of a cystic fibrosis therapeutic agent and a second container filled with a composition comprising a redox-balancing amount of N-acetylcysteine, a pharmaceutically acceptable salt of N-acetylcysteine, or a pharmaceutically acceptable derivative of N-acetylcysteine, and a pharmaceutically acceptable carrier.

In another embodiment, a pharmaceutical kit for treating redox imbalance in cystic fibrosis patients according to the present invention includes a first container filled with a pharmaceutically effective amount of a cystic fibrosis therapeutic agent and a second container filled with a composition comprising a redox-balancing amount of N-acetylcysteine, a pharmaceutically acceptable salt of N-acetylcysteine, or a pharmaceutically acceptable derivative of N-acetylcysteine, and a pharmaceutically acceptable carrier.

In yet another embodiment, a pharmaceutical kit for treating inflammation and redox imbalance in cystic fibrosis patients according to the present invention includes a first container filled with a pharmaceutically effective amount of a cystic fibrosis therapeutic agent and a second container filled with a composition comprising an inflammation-reducing and redox-balancing amount of N-acetylcysteine, a pharmaceutically acceptable salt of N-acetylcysteine, or a pharmaceutically acceptable derivative of N-acetylcysteine, and a pharmaceutically acceptable carrier.

In some embodiments known techniques are used to monitor lung function. Such known techniques include, but are not limited to spirometry, which provides information about airflow limitation and lung volumes; plethysmography, which provides information about airway resistance, total lung size, and trapped gas; transfer factor, which provides information about alveolar function; gas washout tests, which provide information about gas mixing, small airway function, and heterogeneous changes in compliance; computational tomography, which provides information about large and small airway deterioration; and oscillometry, which may provide information about small airways.

In another embodiment of the present invention, compositions and methods of the present invention may be used in combination with known therapeutic agents, provided that they are compatible with each other. “Compatible” as used herein means that the compositions and methods of the present invention are capable of being combined with existing therapies in a manner such that there is no interaction that would substantially reduce the efficacy of either the compositions or methods of the present invention or the therapies under ordinary use conditions.

In some embodiments, existing cystic fibrosis therapeutic agents that may be combined with the compositions and methods of the present invention include, but are not limited to, anti-infective agents, bronchodilating agents, and anti-inflammatory agents.

Lung and airway infections in cystic fibrosis may be treated with potent anti-infective agents, including antibiotics, to improve lung function, reduce days spent in the hospital and to reduce use of intravenous antibiotics to reduce bacterial levels in the lungs. Inhaled antibiotics also are used to prevent lung infections that may lead to hospitalization.

To minimize certain side effects, bronchodilating agents often are used along with inhaled antibiotics. Bronchodilating agents are used widely for treating a variety of obstructive lung diseases, including cystic fibrosis. They relax smooth muscle in the small airways of the lungs, which dilates the airways and makes breathing easier, particularly when airways are narrowed by inflammation. Inhaled bronchodilator medications used in asthma, such as albuterol, have improved breathing in some people with cystic fibrosis. When used to treat cystic fibrosis, bronchodilating agents are usually given through a nebulizer or with a handheld inhaler. Airway dilatation before physiotherapy helps the cystic fibrosis patient to clear chest secretions.

Nonsteroidal anti-inflammatory agents reduce inflammation and pain. Cystic fibrosis patients often have persistent lung inflammation which becomes part of the cycle of continued lung damage in these patients. Anti-inflammatory medications, such as ibuprofen, in some patients with CF help to reduce this inflammation. In some children, anti-inflammatory medications may significantly slow the progression of lung disease and improve breathing.

In some embodiments, therapeutic agents, such as corticosteroids, anticoagulation agents, and pirfenidone, may be administered to treat the inflammation present in some patients with IPF in combination with the compositions and methods of the present invention. Antimicrobial agents also may be used to treat bacterial organisms, opportunistic pathogens, and common respiratory viruses.

In some embodiments, standard doses of existing therapeutic agents for chronic and acute exacerbations of asthma may be combined with the compositions and methods of the present invention. These include, but are not limited to, antimicrobial agents, bronchodilators (e.g., epinephrine, terbutaline, ipratropium (Atrovent®), inhaled corticosteroids, leukotriene antagonists, β-agonists (e.g., albuterol [e.g., Ventolin®, Proventil®, levalbuterol, Metaproterenol Sulfate (Alupent), isoprotenerol, chromolyn sodium; aminophylline, and theophylline.

In another embodiment of the present invention, compositions and methods of the present invention may be used in combination with known therapies, provided that they are compatible with each other.

The term “respiratory therapy” as used herein refers to chest physiotherapy, which is used to help clear excess mucus out of the lungs. To perform chest physiotherapy, a patient is placed in various positions allowing major segments of the lungs to point downward and then clapping firmly over chest and back on part of the lung segment to shake the mucus loose. Once loosened, the mucus will fall to the large airways, where it may be coughed out. Chest physiotherapy may be time-consuming since 3-5 minutes is spent clapping over 10-12 lung segments. It is also difficult for patients to perform on themselves and usually requires a skilled caregiver.

The term “rehabilitative therapy” refers to a therapy designed to help patients use their energy more efficiently, i.e., in a way that requires less oxygen. Rehabilitative therapy improves shortness of breath and overall survival, especially in those with advanced disease.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein may also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. All technical and scientific terms used herein have the same meaning.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Treatment of Cystic Fibrosis Patients with Oral N-Acetylcysteine

A phase I trial of high-dose oral N-acetylcysteine (NAC) in CF has been completed. This CF Foundation-sponsored dose-escalation safety pilot study was designed to assess the dose of oral NAC that may be used safely in order to replenish glutathione (GSH) stores in subjects with CF, with the objectives of restoring a proper redox balance and limiting lung inflammation in patients.

Safety was excellent with all doses tested (1.8, 2.4 and 3.0 g/d, t.i.d, for 4 weeks, N=6 in each cohort). No clinical adverse effect was identified based on physical examination, CBC, laboratory tests, and the CF patient's quality of life (“QOL”). Very mild and infrequent drug-related adverse effects were reported in 6 out of 18 patients (Table 1): heartburn (N=4), nausea (N=1), bad taste (N=1). Doses of 2.4 and 3.0 g/d had less reported adverse effects than 1.8 g/d. Treatment compliance was high (93±1%) and not impacted by drug-related adverse effects (P>0.7) or dose (P>0.3).

With regards to efficacy, very significant positive effects of the treatment were documented. These positive effects (Table 2) included amelioration of: 1—Whole blood GSH (+11%, P=0.03), as measured by HPLC and blood neutrophil GSH (+17%, P=0.03), as measured by flow cytometry; 2—Live sputum leukocyte (−21%, P=0.03) and neutrophil (−25%, P=0.02) counts, as measured by microscopy and sputum elastase activity (−44%, P=0.02), as measured by kinetic spectrophotometry; and 3—Perceived weight gain (P=0.01), as measured by the CF QOL

After excluding three patients without basal lung inflammation (total live leukocytes in sputum in normal range [<0.9, Log 10 scale]), treatment effects were even more pronounced: 1—Whole blood GSH (+14%, P=0.02) and blood neutrophil GSH (+25%, P=0.003); 2—Live sputum leukocyte (−28, P=0.005) and neutrophil (−32%, P=0.003) counts and sputum elastase activity (−46%, P=0.02), as well as % neutrophils in sputum (−9%, P=0.04) and sputum IL-8 (−25%, P=0.02); 3—Perceived weight gain, on the other hand, was less significantly altered (P=0.05) when excluding the three CF patients without basal lung inflammation

The 3 dose cohorts were not significantly different with regards to most outcome measurements, but the second and third dose cohort (2.4 and 3.0 g/d) performed slightly better overall than the first (1.8 g/d). As expected with short-term treatment (4 weeks), Pulmonary Function Testing results (“PFT”) were not changed.

1. Data Acquisition

Data acquisition was completed very satisfactorily for clinical assessment, clinical laboratory tests and research tests. Only one patient in cohort 1 failed to give enough blood to perform both clinical laboratory and research tests so that only the latter were performed.

2. Safety, Adverse Effects and Compliance

Safety assessment did not raise any particular concern. Sputum induction was well tolerated. No clinical adverse effect of treatment was identified based on physical examination, CBC, common laboratory tests and CF QOL (no diarrhea or vomiting recorded). High-dose oral NAC thus was very well tolerated, with only very mild drug-related adverse effects (Table 1, below). Adverse effects were not correlated with dose, patient age, gender, P. aeruginosa status or other parameters. Compliance was excellent, averaging 93±1% (mean±SE) overall and was not influenced by the advent of reported adverse effects and did not differ between the three dose cohorts. Therefore, dose escalation from cohort 1 to 3 proceeded with no safety concerns.

TABLE 1
Safety and compliance

| Subject information | | | | | | Adverse effects | | | |
|---|---|---|---|---|---|---|---|---|---|
| Trial | | Age | | Paer | Compliance | Clinical | Patient | Duration | Probable |
| ID | Cohort | (yrs) | Gender | status | (%) | monitoring | reporting | (days) | cause(s) |
| 001 | 1 | 11 | F | N | 88 | None | Headache | 1 | Dehydration |
| 002 | 1 | 11 | F | Y | 93 | None | Increased cough, | 9 | Infection |
| | | | | | | | sputum; | | |
| | | | | | | | decreased peak | | |
| | | | | | | | flow and | | |
| | | | | | | | exercise | | |
| | | | | | | | tolerance | | |
| 003 | 1 | 40 | F | N | 96 | None | Heartburn | 8 | Drug |
| 004 | 1 | 18 | F | Y | 93 | None | Heartburn | 5 | Drug |
| 005 | 1 | 16 | F | N | 76 | None | Nausea | 3 | Drug |
| 006 | 1 | 32 | F | Y | 96 | None | Heartburn | 19 | Drug |
| 007 | 2 | 14 | F | Y | 87 | None | None | N/A | N/A |
| 008 | 2 | 14 | F | Y | 94 | None | Sore throat | 1 | Infection |
| 009 | 2 | 12 | M | Y | 96 | None | Headache, mild | 28 | Ibuprofen |
| | | | | | | | cough | | withdrawal |
| 010 | 2 | 28 | F | Y | 100 | None | Bad taste | 28 | Drug |
| 011 | 2 | 19 | F | Y | 93 | None | Rash | 3 | Contact |
| | | | | | | | | | dermatitis |
| 012 | 2 | 44 | F | Y | 92 | None | None | N/A | N/A |
| 013 | 3 | 27 | M | Y | 94 | None | Heartburn | 10 | Drug |
| 014 | 3 | 35 | F | Y | 94 | None | Cold symptoms | 1 | Infection |
| 015 | 3 | 38 | M | Y | 95 | None | Constipation | 2 | Ddistal |
| | | | | | | | | | intestinal |
| | | | | | | | | | obstruction |
| | | | | | | | | | syndrome |
| 016 | 3 | 23 | M | N | 93 | None | Mild cough, | 10 | Lung |
| | | | | | | | chest pain | | disease |
| 017 | 3 | 31 | M | Y | 100 | None | Weight loss, | 28 | Lung |
| | | | | | | | mild cough | | disease |
| 018 | 3 | 31 | M | Y | 94 | None | Increased | 18 | N/A |
| | | | | | | | sputum | | |

3. Efficacy

In addition to ascertaining the safety of high-dose oral NAC treatment in CF patients, this pilot phase was also designed to provide preliminary assessment of treatment efficacy on numerous outcome measurements, including:

1. Redox balance, as reflected chiefly by (i) whole blood GSH measured by HPLC, and (ii) live blood neutrophil GSH, measured by flow cytometry

2. Lung inflammation, as reflected chiefly by (i) sputum counts in total live leukocytes and neutrophils (along with % neutrophils in sputum); (iii) plasma/sputum levels of elastase and interleukin-8 (IL-8) measured by spectrophotometry and ELISA

3. Lung function, as measured by spirometry.

Differences between basal and post-NAC values were studied by matched pair analysis, first, without distinguishing dose cohorts, to detect drug effects, and second, with dose cohorts as a factor, in order to detect potential dose effects. Results show that 4 week-treatment with high-dose oral NAC significantly increased the redox balance and reduced lung inflammation.

In addition, analysis of the CF QOL questionnaire revealed a significant effect on perceived weight gain. With regards to lung function, none of the parameters measured by spirometry showed any change, even as important redox and inflammatory parameters were improved upon treatment. This result was expected, based on the power analysis included in our original proposal. Any sizeable change in lung function will likely require longer treatment and larger group size, which we look forward to implementing in the placebo-controlled phase of the study.

Patients with more severe lung inflammation responded better to NAC, notably in terms of the reduction in live sputum leukocytes. In particular, three patients (patients 001, 011, and 016: one in each cohort) were in the normal range of live sputum leukocytes (<0.9 Log 10). When these three patients were excluded, treatment effects were much more significant (Table 2). In addition, other drug effects became significant, e.g., decreases in sputum IL-8 and percent (%) neutrophils.

TABLE 2
Significant drug effects during the phase I trial

| | | Variable | | | | | | | | |
|---|---|---|---|---|---|---|---|---|---|---|
| | | Whole | | Live | Live | Neutrophils | | Elastase | Perceived | |
| | | blood | Neutrophil | sputum | sputum | sputum | IL-8 in | in | weight | |
| Subjects | Statistics | GSH | GSH | leukocytes | neutrophils | (%) | sputum | sputum | gain | FeV1 |
| All | Change | +11% | +17% | −21% | −25% | NS | NS | −44% | Increased | NS |
| (N = 18) | P value | 0.03 | 0.03 | 0.03 | 0.02 | | | 0.02 | 0.01 | |
| 3 | Change | +14% | +25% | −28% | −32% | −9% | −25% | −46% | Increased | NS |
| patients | P value | 0.02 | 0.0003 | 0.005 | 0.003 | 0.04 | 0.02 | 0.02 | 0.05 | |
| excluded | | | | | | | | | | |
| (N = 15) | | | | | | | | | | |

Except for baseline sputum count, the drug effect as measured through all the above variables was not dependent on any of the baseline parameters and was not significantly dependent on dose. However, dose cohort 2 (and to a lesser extent cohort 3) showed significant drug effects on additional selected parameters (for example, absolute numbers of neutrophils in blood, which was significantly decreased by 27%), which was more likely related to lower baseline conditions than to a dose effect per se. Indeed, cohort 2 was more severely affected with regards to several surrogate markers of disease prior to treatment (lower FEV1, all infected with P. aeruginosa, lower perceived weight gain). Thus, cohort 2 may have been more conducive to revealing drug effects than the other two cohorts.

Systemic redox-based therapy is an attractive idea for CF, since redox imbalance is a well-recognized aspect of the disease, yet seldom considered as a bona fide therapeutic target. In that context, the safety and efficacy of high-dose oral NAC on redox parameters, inflammation and lung function has been assessed in CF patients. The results of the phase I trial show that NAC in oral doses as high as 3.0 g/d do not cause any safety concerns when administered for as long as 4 weeks, thus confirming previous studies in other diseases. The phase I trial also provides strong evidence that high-dose oral NAC may significantly ameliorate both systemic redox stress and lung inflammation in CF.

### Example 2

### Placebo-Controlled Phase of the CF Trial

Summary. Based on the success of the phase I trial, the trial proceeded to phase II. This single-center trial consisted of a 12-week placebo-controlled section followed by a 12-week open label section, with oral NAC 0.9 g, taken three times daily. The statistical plan for the study was designed to assess the safety and efficacy of NAC versus placebo, at 0 week and 12-week timepoints (placebo-controlled section). Of the 24 subjects screened for eligibility, 21 were enrolled and randomized into NAC and placebo groups. One subject asked to be withdrawn from the prior to the 6 week time point because the medication regimen was too onerous. The subject failed to return for the 6-week time point or for the final study visit at week 12. Two other subjects also were removed from participation in the study by the principal investigator due to poor adherence to the study protocol. These subjects did not return for either the 6- or the 12-week study visits. Thus, 18 subjects are included in this intent-to-treat (ITT) analysis (9 on NAC and 9 on placebo).

Both NAC and placebo were very well tolerated and did not cause any serious adverse events. Adverse events were all mild and did not affect adherence to treatment, which was consistently high, aside from the three subjects mentioned above (>93%). Of the 18 subjects included in the ITT analysis, two reported symptoms of daily indigestion related to drug intake. One of these subjects completed the 12-week treatment period with 95% of study drug compliance, but the other patient was removed from the study due to 26% compliance rate discovered by the study coordinators prior to the 6-week follow-up.

In phase 1, NAC treatment decreased sputum neutrophil count and extracellular human neutrophil elastase (HNE) activity. In this phase 2 trial, the NAC group, but not the placebo group, showed significant decreases in sputum neutrophil count (primary endpoint), blood neutrophil GSH and sputum HNE enzymatic activity (secondary endpoints), as well as sputum HNE and interleukin-8 protein levels. No significant effect was measured for the functional expiratory volume in 1 second as a percent of predicted for age (FEV1% pred.) (a secondary endpoint in this study). Of note, pulmonary exacerbations (which were not a primary outcome measure for this study) were significantly less frequent in the NAC group (2/9) than in the placebo group (7/9 subjects).

Serious adverse events and adverse events. During this phase 2 trial, only one SAE was reported. Subject #2011, who suffered acute pyelonephritis, had a previous history of recurrent urinary tract infections and had had a urinary tract infection the month prior. This SAE occurred 5 days after the subject received the first dose of NAC. The subject was admitted to a local hospital and was treated for 5 days with IV Levaquin and prednisone and discharged 5 days after admission to the hospital. The subject reported that she did not take the study drug during hospitalization but resumed taking the study drug right after hospitalization. The subject did not report for evaluation at the six week time point and was the removed from the study. This SAE was not considered related to the study drug. No other SAEs were reported for the remainder of the placebo-controlled section. Only one subject out of 18 reported adverse events that were likely to be related to the study drug (or placebo). This subject (#2012) reported daily abdominal discomfort/indigestion through the study, which was efficiently treated by Pepcid AC and did not lead to decreased adherence to treatment. There was no other consistent gastrointestinal (GI) complaint related to NAC or placebo. No specific pattern of adverse events emerged from this phase 2 study, confirming the phase 1 safety data. CF QOL questionnaires showed a significant reduction in flatulence observed in the NAC group, but not in the placebo group. This may represent a potential positive effect on the digestive abnormalities of CF subjects, especially as NAC is a known remedy for treatment of DIOS in CF patients. As used herein, the term “DIOS”, which stands for “Distal Intestinal Obstruction Syndrome” refers to a condition unique to CF that occurs due to the accumulation of viscous mucous and fecal material in the terminal ileum, caecum and ascending colon, which may cause progressive symptoms of recurrent colicky abdominal pain, bloating, nausea and anorexia, and signs of small intestinal obstruction. No other changes were seen as per the CF QOL. Complete blood count and chemistry parameters were not affected by 12-week NAC/placebo treatment, except for marginal changes in red blood cell distribution width and calcium in the NAC group. None of these changes led to values outside of the normal range. No change in liver enzymes was noted. This data confirms the lack of toxicity of high-dose oral NAC in CF.

Intention-to-treat analysis of efficacy endpoints. Besides the necessary assessment of the safety of high-dose oral NAC in a placebo-controlled setting, this phase 2 trial also was designed to gain a better understanding of treatment efficacy with regards to improving inflammation, redox imbalance and lung function in CF, albeit within the limits inherent to a small study. In particular, the study looked to confirm the positive effects of high-dose oral NAC seen on sputum neutrophil count and HNE activity obtained in phase 1. The primary efficacy endpoint in this phase 2 study is sputum neutrophil count (based on the quantification of live neutrophils by microscopy, reflecting lung inflammation) and the four secondary efficacy endpoints are: (i) FEV1 (% Pred), reflecting lung function; (ii) blood neutrophil GSH, reflecting systemic redox imbalance; (iii) sputum HNE activity, reflecting lung inflammation, the current best predictor of CF lung disease; and (iv) whole blood GSH, reflecting systemic redox imbalance. Data on all other main efficacy endpoints (along with sputum HNE and IL-8 protein levels as additional indicators of inflammation) is presented in Table 3 (below) for all 9 subjects of the NAC group and 9 subjects in the placebo group included in the ITT analysis.

TABLE 3
ITT analysis of main efficacy endpoints (placebo-controlled section).

| Endpoint | Type | Group | Value wk 0 | Value wk 12 | P within group | P between groups |
|---|---|---|---|---|---|---|
| Sputum neutrophil count | Inflammation | NAC | 1.41 ± 0.17 | 1.24 ± 0.18 | 0.03 | 0.85 |
| (Log10) | | Placebo | 1.05 ± 0.18 | 0.81 ± 0.23 | 0.22 | |
| | | P between groups | 0.15 | 0.16 | | |
| Functional expiratory | Lung function | NAC | 73.7 ± 7.6 | 75.6 ± 8.2 | 0.15 | 0.74 |
| volume in 1s (% Pred) | | Placebo | 69.3 ± 8.3 | 69.7 ± 8.3 | 0.47 | |
| | | P between groups | 0.70 | 0.62 | | |
| Sputum HNE enzymatic | Inflammation | NAC | 3.61 ± 0.15 | 3.16 ± 0.20 | 0.006 | 0.39 |
| activity (Log10) | | Placebo | 3.08 ± 0.19 | 2.87 ± 0.18 | 0.20 | |
| | | P between groups | 0.04 | 0.30 | | |
| Blood neutrophil intracellular | Redox | NAC | 4.04 ± 0.08 | 4.10 ± 0.10 | 0.02 | 0.60 |
| GSH | | Placebo | 4.00 ± 0.07 | 4.04 ± 0.07 | 0.22 | |
| | | P between groups | 0.71 | 0.59 | | |
| Sputum HNE protein levels | Inflammation | NAC | 0.04 ± 0.13 | −0.27 ± 0.12 | 0.04 | 0.66 |
| (Log10) | | Placebo | −0.51 ± 0.15 | −0.69 ± 0.22 | 0.21 | |
| | | P between groups | 0.01 | 0.11 | | |
| Sputum IL-8 protein levels | Inflammation | NAC | 2.01 ± 0.12 | 1.81 ± 0.18 | 0.03 | 0.70 |
| (Log10) | | Placebo | 1.68 ± 0.10 | 1.35 ± 0.30 | 0.17 | |
| | | P between groups | 0.06 | 0.21 | | |

Consistent with the phase 1 results, sputum neutrophil count, sputum HNE enzymatic activity, sputum HNE levels, and IL-8 levels were significantly decreased in the NAC group but not in the placebo group. These various markers of inflammation were measured independently with different methodologies (e.g., microscopy, kinetic spectrophotometry, enzyme-linked immunosorbent assay), the results of which further strengthen the significance of these positive outcomes. Moreover, blood neutrophil GSH was significantly increased in the NAC group but not in the placebo group, confirming the possible causative link between low GSH levels in CF blood neutrophils and their increased propensity to migrate into and subsequently damage the patients' lungs. The ITT analysis showed no significant decline in pulmonary function tests (PFTs) over the course of the trial, which confirms the safety of the treatment regimen. However, the analysis also failed to detect any significant improvement of FEV1 (% Pred) or other measures of lung function (data not included) in the NAC group. PFTs are notoriously weak endpoints in CF trials due to issues with lack of sensitivity. The low number of subjects and the confounding effect of concurrent high-impact treatments (such as antibiotics or corticosteroid) on the evaluation of PFTs also contributed to decrease the likelihood of measuring significant changes in this first phase 2 trial. Between-group analysis of pre- vs. post-treatment data failed to return significant values for any of the above endpoints. This also likely is due to the low number of subjects in this first phase 2 trial and to the confounding effect of concurrent high-impact treatments on endpoint evaluation.

Rationale for future studies. Our phase 1 data and phase 2 data presented here establish an excellent safety profile for high-dose oral NAC treatment in CF patients. Both sets of data also strongly suggest a positive effect of high-dose oral NAC on lung inflammation and systemic redox imbalance. Without being limited by theory, by reducing the amount of blood neutrophils in CF lungs, high-dose oral NAC may affect positively the local conditions that normally lead to progressive lung function decline, notably the amount of extracellular HNE enzymatic activity in CF lungs. An upcoming phase 2b trial will assess the effect of high-dose oral NAC on CF PFTs.

### Example 3

### Use of NAC to Treat Acute Exacerbations of CF

A CF patient showing the symptoms of an acute exacerbation of CF (including, but not limited to, increased respiratory symptoms, reduction in forced expiratory volume in one second (FEV1) of more than 10%, and a decision to treat with intravenous antibiotics) may be treated with a composition comprising an acute exacerbation-reducing amount of either the purified L-enantiomer or the racemate mixture composed of equal proportions of the D- and L-isomers of NAC administered either serially or co-administered two, three or four times a day up to the highest tolerable dose, given that there will be individual variability in the ability to tolerate NAC. This dosage of NAC is sufficient to decrease key aspects of an acute exacerbation of CF in such patients.

The phase 2a data suggest that chronic high-dose oral NAC treatment may potentially decrease the number of sinus and lung exacerbations in CF patients. During week 0 through week12, exacerbations of sinus/lung disease affected 9/18 subjects. Subjects were less prone to exacerbations in the NAC than in the placebo group (2/9 vs. 7/9, respectively, P=0.04, Fisher's exact test). A key molecular correlate of exacerbations, namely plasma levels of the cytokine interleukin-17 (IL-17) also was decreased in the NAC group compared to the placebo-group (P=0.02), further confirming the anti-inflammatory effect of NAC in CF and corroborating its positive effect on acute attacks. IL-17 recently has been identified as a potent T-cell derived modulator of acute neutrophilic lung inflammation [Linden, A., et al. Neutrophils, interleukin-17A and lung disease. Eur. Respir. J. 25:159-172 (2008)]

### Example 3

### Use of NAC to Treat Acute Exacerbations of IPF

A patient showing the symptoms of an acute exacerbation of IPF (including, but not limited to, idiopathic acute respiratory deterioration) may be treated with a composition comprising an acute exacerbation-reducing amount of either the purified L-enantiomer or the racemate mixture composed of equal proportions of the D- and L-isomers of NAC administered either serially or co-administered two, three or four times a day up to the highest tolerable dose, given that there will be individual variability in the ability to tolerate NAC. This dosage of NAC is sufficient to decrease key aspects of an acute exacerbation of IPF in such patients.

### Example 4

### Use of NAC to Treat Acute Exacerbations of Asthma

A child or adult showing the symptoms of an acute exacerbation of asthma (including, but not limited to, a sudden increase in breathlessness over the preceding 48 hours and presence of one of the following signs: tachypnea (respiratory rate of >18), use of accessory muscles or respiration, audible wheezing, prolonged expiration with rhonchi on ausculation or a silent chest) may be treated with a composition comprising at least one standard asthma therapeutic agent and an acute exacerbation-reducing amount of either the purified L-enantiomer or the racemate mixture composed of equal proportions of the D- and L-isomers of NAC administered either serially or co-administered two, three or four times a day up to the highest tolerable dose, given that there will be individual variability in the ability to tolerate NAC. This dosage of NAC is sufficient to decrease key aspects of an acute exacerbation of asthma in such patients.

### Example 5

### Use of NAC to Treat Acute Exacerbations of TB in HIV Patients

An HIV patient having latent or active TB who is being treated with a formulation comprising a therapeutically effective amount of a multi-drug regimen as normally used to treat HIV and/or TB may be further treated with a composition comprising an acute exacerbation reducing amount of either the purified L-enantiomer or the racemate mixture composed of equal proportions of the D- and L-isomers of NAC administered either serially or co-administered two, three or four times a day up to the highest tolerable dose, given that there will be individual variability in the ability to tolerate NAC. This dosage of NAC is sufficient to decrease key aspects of IRIS in such patients.

While the present invention has been described with reference to the specific embodiments thereof, it should be understood by those skilled in the art that various changes may be made and equivalents may be substituted without departing from the true spirit and scope of the Invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating at least one symptom of an acute exacerbation of an inflammatory lung disease other than COPD in a patient in need thereof, the method comprising the step of: (a) administering to a patient in need thereof a pharmaceutical composition comprising (1) an acute exacerbation-reducing amount of N-acetylcysteine, a pharmaceutically acceptable salt of N-acetylcysteine, or a pharmaceutically acceptable derivative of N-acetylcysteine, and (2) a pharmaceutically acceptable carrier, and thereby modulating the symptoms of the acute exacerbation.

2. The method according to claim 1, wherein the inflammatory lung disease is cystic fibrosis.

3. The method according to claim 1, wherein the inflammatory lung disease is an interstitial lung disease.

4. The method according to claim 3, wherein the interstitial lung disease is idiopathic pulmonary fibrosis.

5. The method according to claim 1, wherein the inflammatory lung disease is asthma.

6. The method according to claim 1, wherein the inflammatory lung disease is tuberculosis and the patient is an HIV patient.

7. The method according to claim 1, wherein in step (a) of the method the pharmaceutical composition is administered systemically by a route selected from the group consisting of orally, buccally, topically, by inhalation, by insufflation, parenterally and rectally.

8. The method according to claim 1, wherein in step (a) of the method, the pharmaceutical composition is administered orally.

9. The method according to claim 1, wherein the acute exacerbation-reducing amount of N-acetylcysteine, a pharmaceutically acceptable salt of N-acetylcysteine, or a pharmaceutically acceptable derivative of N-acetylcysteine in the pharmaceutical composition administered orally is about 1.8 grams per day to about 6 grams per day, and less than or equal to 200 mg per kg per day.

10. The method according to claim 1, wherein the acute exacerbation-reducing amount of N-acetylcysteine, a pharmaceutically acceptable salt of N-acetylcysteine, or a pharmaceutically acceptable derivative of N-acetylcysteine in the pharmaceutical composition administered orally is at least about 1800 mg per day and less than or equal to 200 mg per kg per day.

11. The method according to claim 1, wherein the acute exacerbation-reducing amount of N-acetylcysteine, a pharmaceutically acceptable salt of N-acetylcysteine, or a pharmaceutically acceptable derivative of N-acetylcysteine in the pharmaceutical composition administered orally is at least about 2400 mg per day and less than or equal to 200 mg per kg per day.

12. The method according to claim 1, wherein the acute exacerbation-reducing amount of N-acetylcysteine, a pharmaceutically acceptable salt of N-acetylcysteine, or a pharmaceutically acceptable derivative of N-acetylcysteine in the pharmaceutical composition administered orally is at least about 3000 mg per day and less than or equal to 200 mg per kg per day.

13. The method according to claim 1, wherein in step (a) of the method, the pharmaceutical composition is administered parenterally.

14. The method according to claim 13, wherein the acute exacerbation-reducing amount of N-acetylcysteine, a pharmaceutically acceptable salt of N-acetylcysteine, or a pharmaceutically acceptable derivative of N-acetylcysteine in the pharmaceutical composition administered parenterally is about 200 mg NAC to about 2000 mg NAC per dosage unit.

15. The method according to claim 1, wherein the method further comprises the step of (b) administering a pharmaceutically effective amount of a disease-specific therapeutic agent.

16. The method according to claim 15, wherein the disease specific therapeutic agent comprises at least one cystic fibrosis therapeutic agent selected from the group consisting of an anti-infective agent, a bronchodilating agent, and an anti-inflammatory agent.

17. The method according to claim 15, wherein the disease-specific therapeutic agent comprises at least one idiopathic pulmonary fibrosis therapeutic agent selected from the group consisting of a corticosteroid agent, an anticoagulation agent, pirfenidone, and an antimicrobial agent.

18. The method according to claim 15, wherein the disease-specific therapeutic agent comprises at least one asthma therapeutic agent selected from the group consisting of an antimicrobial agent, a bronchodilator agent, a corticosteroid; a leukotriene antagonist; and a agonist.

19. The method according to claim 15, wherein the disease specific therapeutic agent comprises at least one tuberculosis therapeutic agent.

20. The method according to claim 1, the method further comprising the step of (b) administering a respiratory therapy to the patient.

21. The method according to claim 1, the method further comprising the step of (b) administering a rehabilitation therapy to the patient.

22. A pharmaceutical kit for treating an acute exacerbation of an inflammatory lung disease in a subject in need thereof, the kit comprising a) a first container containing a pharmaceutically effective amount of a disease-specific therapeutic agent, and b) a second container containing a pharmaceutical composition comprising (i) an acute exacerbation-reducing amount of N-acetylcysteine, a pharmaceutically acceptable salt of N-acetylcysteine, or a pharmaceutically acceptable derivative of N-acetylcysteine, and (ii) a pharmaceutically acceptable carrier.

23. The pharmaceutical kit according to claim 23, wherein the disease specific agent in the first container comprises at least one cystic fibrosis agent selected from the group consisting of an anti-infective agent, a bronchodilating agent, and an anti-inflammatory agent.

24. The pharmaceutical kit according to claim 23, wherein the disease-specific agent in the first container comprises at least one idiopathic pulmonary fibrosis therapeutic agent selected from the group consisting of a corticosteroid agent, an anticoagulation agent, pirfenidone, and an antimicrobial agent.

25. The pharmaceutical kit according to claim 23, wherein the disease-specific agent in the first container comprises at least one asthma therapeutic agent selected from the group consisting of an antimicrobial agent, a bronchodilator agent, a corticosteroid; a leukotriene antagonist; and a β-agonist.

26. The pharmaceutical kit according to claim 23, wherein the disease specific agent comprises at least one tuberculosis therapeutic agent.
